(19) United States
(12) Patent Application Publication
(10) Pub. No.: US 2022/0023168 A1
(43) Pub. Date: Jan. 27, 2022

(54) ANTIMICROBIAL COSMETIC PREPARATION

(71) Applicant: HYDRODYNE SYSTEMS LTD, Chesterfield (GB)

(72) Inventor: Alan Taylor, Chesterfield (GB)

(21) Appl. No.: 17/380,884

(22) Filed: Jul. 20, 2021

(30) Foreign Application Priority Data
Jul. 21, 2020 (GB) 2011238.9

(51) Int. Cl. A61K 8/19; A61K 8/73; A61Q 17/00

(52) U.S. Cl. CPC A61K 8/19; A61K 8/736; A61K 2800/52; A61K 2800/48; A61Q 17/005

(57) ABSTRACT

A water-based anti-microbial cosmetic preparation comprising 0.025% to 3.2% of silver-chitosan dispersion by weight and not more than 10% of a non-ionic surfactant by weight. The preparation has a ratio of non-ionic surfactant to silver chitosan by weight of at least 0.2:1 to allow the silver chitosan to form a film on the surface of the skin. The preparation has a pH of between 3 and 6.5 to ensure that the silver chitosan to ensure that the preparation is tolerable on the skin and to ensure that the silver does not precipitate. The preparation does not contain an ionic surfactant in order to avoid precipitating the silver chitosan out of the preparation.

### CROSS REFERENCE TO RELATED APPLICATIONS

The present application claims priority to and the benefit of Taylor, United Kingdom Patent Application No. GB 2011238.9, filed Jul. 21, 2020, and entitled “An Antimicrobial Cosmetic Preparation.” The entire contents of this application are incorporated herein by reference.

### FIELD OF INVENTION

The present invention relates to cosmetic preparations that are applied directly to the skin such as cleansers, foundations, and serums.

### BACKGROUND TO THE INVENTION

A wide range of cosmetic preparations are applied to the face and body for health and beauty reasons. Examples of commonly used facial cosmetic preparations include cleansers, toners, serums, primers, face creams, and foundations. These cosmetic preparations are typically applied in a uniform layer over substantially the whole of the surface of the face. Hand creams and emolients are also used and applied in a uniform layer of substantially the whole of the surface of the hands.

Silver chitosan is a known powder or dispersion consisting of chitosan and/or its derivatives as a carrier, dispersant, and protective agent for nano silver. Silver chitosan powders and dispersions provide a stabilised form of nano silver that can be used for antimicrobial applications. Nano silver stabilised as atomic silver bonded to the amine groups in recurring chitosan groups has strong bacteriostatic, bactericidal and broad-spectrum antibacterial activities, and due to the quantum effect, small size effect, and great specific surface area, it has an antibacterial effect unmatched by traditional inorganic antibacterial agents.

Silver chitosan powders and dispersions are well known and has been developed for various applications including wound dressings, anti-microbial hydrogels and other medical applications. Silver chitosan powders and dispersions have also been applied to various fabrics to produce antimicrobial fabrics. EP1313372 discloses a method of preparing silver chitosan powder, including optionally applying the resulting powder to fabrics.

Many cosmetic preparations now include antimicrobial components both to protect against the growth of microorganisms in the cosmetic preparation itself and to treat skin to which the preparation is applied. It is believed that the inclusion of antimicrobial components in cosmetic preparations can assist in killing harmful bacteria present on the skin, preventing the growth of unwanted microorganisms, and can aid in the treatment of conditions such as acne and skin inflammation. Current antimicrobial components used in cosmetic preparations include organic acids, potassium sorbate, and silver compounds. Silver compounds that are currently used include silver carbonate, silver sulphate, and silver citrate. None of these silver compounds reliably provide nano silver in a cosmetic preparation.

There have been to prepare cosmetic preparations comprising silver chitosan, in order to provide a preparation which readily provides nano-silver, but these have been largely unsuccessful. Therefore, there is a desire to produce a cosmetic preparation comprising silver chitosan that is long-lasting and effective.

### SUMMARY OF THE INVENTION

The present invention provides a water based anti-microbial cosmetic preparation comprising:
0.025% to 3.2% of silver-chitosan dispersion by weight;
not more than 10% of non-ionic surfactant by weight; and

wherein:
a ratio of non-ionic surfactant to silver chitosan by weight of at least 0.2:1;
the preparation has a pH of between 3 and 6.5; and
the preparation does not contain an ionic surfactant.

The preparation of the present invention is advantageous in that it readily forms a film on the skin of a user. In particular, the combination of the non-ionic surfactant and the silver chitosan dispersion results in the ability of the cosmetic preparation to form a film on the surface of skin when applied.

The silver chitosan dispersion of the present invention can be provided by forming the preparation of between 5% to 80% of a water-based silver chitosan dispersion having a concentration between 0.5% and 4%.

The preparation does not contain an ionic surfactant as the presence of such compounds disrupt the amine bond present in the silver chitosan. In particular, silver bonds to the ionic tail of an ionic surfactant to effect a silver precipitation causing the preparation to discolour with precipitated silver. This is clearly undesirable in most cosmetic preparations. In contrast, non-ionic surfactants allow the silver chitosan to form a film on the surface of the skin without disrupting the amine bond in the silver chitosan. It is important that a cosmetic preparation according to the present invention does not comprise any ionic components that would result in silver precipitation. This is true even an ionic component is not intended to be a surfactant.

Non-ionic surfactants are compounds that comprise a hydrophilic head group and a hydrophobic tail group and that do not carry a charge when in solution. Suitable non-ionic surfactants include, but are not limited to, fatty alcohol ethoxylates, alkyl phenol ethoxylates, and fatty acid alkoxylates. It is understood that the skilled person would understand which non-ionic surfactants are suitable for use in cosmetic preparations. One example of a suitable non-ionic surfactant is alcohol ethoxylate. The composition of the present invention may comprise one or more non-ionic surfactants.

In cosmetic preparations according to the present invention the ratio of non-ionic surfactant to silver chitosan by weight is at least 0.2:1. That is, the minimum ratio of non-ionic surfactant to silver chitosan by weight is 0.2 g of non-ionic surfactant for each 1 g of silver chitosan. For example, for 100 g of 1% silver chitosan solution there must be at least 0.2 g of non-ionic surfactant. Amounts of non-ionic surfactant below a 0.2:1 ratio with silver chitosan provide poor wetting and do not provide a reliable film of the surface of the skin.

Advantageously, the ratio of non-ionic surfactant to silver chitosan by weight is at least 1.5:1, preferably 2:1. A ratio of at least 1.5:1, preferably 2:1, provides excellent wetting of both skin and other surfaces, both porous and non-porous.

The maximum amount of non-ionic surfactant in a cosmetic preparation according to the present invention is 10% of weight. That is, for 100 g of a cosmetic preparation according to the present invention there should be no more than 10 g of non-ionic surfactant. Amounts of non-ionic surfactant above 10% by weight adversely affect the feel of the preparation, making the preparation feel “slippy”.

Silver chitosan is an advantageous antibacterial component of a cosmetic preparation in that it is a mucoadhesive. This means that the silver chitosan readily adheres to bacteria and other similar organic bodies, enhancing its antibacterial activity.

The pH of the cosmetic preparation of the present invention is between 3 and 6.5. This is advantageous in that a pH of below 3 may be irritating to the skin of a user. A pH of above 6.5 increases the risk of the silver chitosan precipitating and discolouring the cosmetic preparation. It is difficult to form films of silver chitosan in cosmetic preparations having a pH above 7 and even if such films can be formed they are turgid and uneven.

Many cosmetic preparations comprising silver chitosan and non-ionic surfactants will have a pH between 3 and 6.5. However, some preparations may normally have a pH outside of this range and in such preparations it may be necessary for the preparation to additionally include a pH modifier in order to bring the pH within the range of 3 and 6.5. Suitable pH modifiers will be apparent to the person skilled in the art.

Generally, cosmetic preparations according to the present invention will not comprise high pH additives such as sodium hydroxide, sodium bicarbonate, potassium hydroxide, triethanolamine, sodium phosphate, L-arginine, and high pH oils and fragrances. Nevertheless it may be possible to include such high pH additives in a cosmetic preparation according to the present invention provided one or more low pH additives are additionally used to maintain the pH of the preparation between 3 and 6.5

The silver chitosan dispersion of the present invention may have a concentration of between 0.5% and 4% by weight. 4% is the maximum content of silver chitosan in water that remains dispersed before precipitating out. 0.5% is the minimum content that allows a film to be reliably formed.

The maximum of water-based silver chitosan dispersion that can be incorporated into a cosmetic preparation according to the present invention is 80% of the total preparation at a maximum content of 4% silver chitosan. Any amount higher than this will result in a discoloured preparation that would not be able to be used.

A cosmetic preparation according to the present invention may comprise any suitable additive or excipient that does not act to precipitate out the silver chitosan and providing the resulting cosmetic preparation maintains a pH between 3 and 6.5. Suitable excipients include but are not limited to perfumes, oils, viscosity modifiers, non-ionic emulsifiers, glycerine, thickeners, and emulsion stabilisers. Generally, any excipient should not be ionic such that it does not disrupt the amine bond present in the silver chitosan to effect silver precipitation.

The cosmetic preparation of the present invention may be any suitable cosmetic preparation. Advantageously, the cosmetic preparation may be a facial preparation such as a cleanser, a toner, a serum, a primer, a face cream, or a foundation. Alternatively, the cosmetic preparation may be a hand or skin preparation such as a hand cream or an emollient.

If the cosmetic preparation according to the present invention is a facial preparation or a hand preparation intended for spreading over substantially the whole of the surface of the hand or the face then it is advantageous that the preparation contains sufficient silver chitosan to provide a uniform film over the surface of the skin to which the preparation is applied.

The approximate surface area of a face or a pair of hands is 300 cm². The minimum thickness of film that can be formed by silver chitosan is approximately 10 nm (0.01 μm). The amount of silver chitosan in the composition should be sufficient to form a film of this thickness. The actual amount required in any particular composition will depend on the amount of the preparation that is intended to be applied in a single application. It is anticipated that the skilled person will be able to determine an appropriate minimum amount of silver chitosan to form a film on the surface of the hands or face.

As an example, 1 ml of a silver chitosan in a 1% aqueous dispersion by weight applied to an area of 300 cm² has been found to give a film thickness of approximately 0.04 μm. 20 ml of a silver chitosan in an 1% aqueous dispersion by weight applied to an area of 300 cm² has been found to provide a film thickness of approximately 1.0 μm. It is anticipated that most cosmetic preparations according to the present invention will be formulated to provide a film of 0.04 μm and 0.20 μm for each application.

When a silver chitosan dispersion is incorporated into a cosmetic formulation with cosmetic components this can affect the ability of the silver chitosan to form films due to the cosmetic components drying at different rates to silver chitosan dispersion. For example, a 1% silver chitosan dispersion is incorporated into a cosmetic preparation with approx. 25% by weight of cosmetic cosmetics and the silver chitosan 1% aqueous dispersion comprises 25% of the cosmetic preparation then a 1 ml application of the cosmetic preparation is expected to give between 1 μm and 4 μm total dried film thickness over a 300 cm² skin surface.

As set out above, chitosan is an excellent mucoadhesive polymer with excellent adhesion properties with the surface epithelial cells of the epidermis. This allows the chitosan of the present invention to act as an excellent base adhesive for other cosmetic components and can act to bind these other components to the skin surface.

Further features and advantages of the present invention will be apparent from the specific examples set out below.

### EXAMPLE 1

The preparation of an antimicrobial hand cream according to the present invention is set out below.

Step 1

A first mixture consisting of:
100 g of 1% silver chitosan dispersion;
1 g phenoxyethanol ethylhexylglycerin (preservative); and
3 g alcohol ethoxylate (non-ionic surfactant)

is prepared and mixed thoroughly in a first container.

Step 2

A second mixture consisting of:
0.5 g xanthan gum (thickener);
30 g Sunflower oil (base); and
3 g glyceryl stearate (non-ionic surfactant)

is prepared and mixed thoroughly in a second container.

Step 3

The first mixture and the second mixture are combined, mixed thoroughly, and heated to a temperature of 70° C. to produce a third mixture.

Step 4

A fourth mixture consisting of
20 g cetearyl alcohol (emulsion stabiliser); and
2 g essential oil fragrance (perfume)

is prepared and mixed thoroughly in a third container.

Step 5

The fourth mixture is added to the third mixture and the combination is mixed until it cools and thickens to produce the anti-microbial hand cream.

This produces rich antimicrobial hand cream for use in treatment of cold weather dermatitis. The resulting hand cream has a pH of 4, which prevents the precipitation of the silver chitosan but is also well tolerated by the skin.

The ratio of non-ionic surfactant to silver chitosan by weight is 3:1. In particular, the composition contains 1 g of silver chitosan and 3 g of alcohol ethoxylate.

### EXAMPLE 2

The following ingredients are mixed together to form a serum according to the present invention

Water −63% by weight of the total finished product

1% silver chitosan dispersion to form 22% by weight

Glycerine 3.4%

Cetyl and Cetearyl Alcohol [non-ionic emulsifiers] 1.5%

Glyceryl Stearate [non-ionic surfactant] 1.7%

Cyclopentasiloxane [Oil for skin texture] 3.4%

Polyethylene Glycol Stearate also known as PEG 100 [non-ionic emulsifier] 1%

Dimethicone and Caprylic Triglyceride [Viscosity modifiers] 3.4%

Fragrances and Shea Butter for texture 0.6%.

### EXAMPLE 3

The components of an antimicrobial serum according to the present invention are shown in the tables below. The silver chloride and chitosan provide the silver chitosan of the present invention whilst the Ceteareth-20 and the cetearyl alcohol are non-ionic surfactants.

| No. | substance | w/w | CAS No. |
|---|---|---|---|
| 1 | Aqua (water) | 75.800 | 7732-18- |
| | | | 5 |
| 2 | Cyclopentasiloxane | 4.000 | 541-02-6 |
| 3 | Caprylic Triglyceride | 4.000 | 65381- |
| | | | 09-1 |
| 4 | Glycerin | 4.000 | 56-81-5 |
| 5 | Dimethicone | 2.000 | 9006-65- |
| | | | 9 |
| 6 | Glyceryl Stearate | 2.000 | 123-94-4 |
| 7 | Ceteareth-20 | 1.750 | 68439- |
| | | | 49-6 |
| 8 | Cetearyl Alcohol | 1.750 | 8005-44- |
| | | | 5 |
| 9 | PEG-100 Stearate | 1.500 | 9004-99- |
| | | | 3 |
| 10 | Triethanolamine | 0.900 | 102-71-6 |
| 11 | Phenoxyethanol | 0.550 | 122-99-6 |
| 12 | Ethylhexylglycerin | 0.550 | 70445- |
| | | | 33-9 |
| 13 | utyrospermum Parkii (Shea) Butter | 0.500 | 91080- |
| | | | 23-8 |
| 14 | Lactic Acid | 0.200 | 50-21-5 |
| 15 | Chitosan | 0.180 | 9012-76- |
| | | | 4 |
| 16 | Limonene | 0.172 | 5989-27- |
| | | | 5 |
| 17 | Allantoin | 0.100 | 97-59-6 |
| 18 | Citrus Tangerina (Tangerine) Peel Oil | 0.050 | 223748- |
| | | | 44-5 |
| 19 | Citrus Aurantium Bergamia (Bergamot) | 0.050 | 8007-75- |
| | Fruit Oil | | 8 |
| 20 | Citrus Grandis (Grapefruit) Peel Oil | 0.050 | 8016-20- |
| | | | 4 |
| 21 | Citrus Aurantium Dulcis (Orange) Peel | 0.050 | 8008-57- |
| | Oil | | 9 |
| 22 | Citral | 0.009 | 5392-40- |
| | | | 5 |
| 23 | Linalool | 0.009 | 78-70-6 |
| 24 | Silver Chloride | 0.003 | 7783-90- |
| | | | 6 |
| 25 | Citronellol | 0.000 | 106-22-9 |
| 26 | Geraniol | 0.000 | 106-24-1 |

| | substance | Purpose |
|---|---|---|
| 1 | Aqua | Water as the main cosmetic base |
| 2 | Cyclopentasiloxane | Silicon lubricant and solvent for other |
| | components | |
| 3 | Caprylic Triglyceride | Emollient and non ionic dispersing agent |
| 4 | Glycerin | Skin smoothing agent |
| 5 | Dimethicone | Viscoelastic agent and viscosity modifier |
| 6 | Glyceryl Stearate | Non ionic Emulsifier and emollient skin |
| | | softening agent |
| 7 | Ceteareth-20 | Non ionic surfactant used as thickening agent |
| 8 | Cetearly Alcohol | Non ionic surfactant for oil/water binding |
| 9 | PEG-100 Stearate | Non ionic emulsifier for oil in water complexes |
| 10 | Triethanolamine | Non ionic surfactant used as a pH buffer to raise |
| | | pH |
| 11 | Phenoxyethanol | Preservative and Biocide |
| 12 | Ethylhexylglycerin | Non ionic preservative usually blended with |
| | | phenoxyethanol |
| 13 | utyrospermum Parkii (Shea) | primarily acts as an emollient and |
| | Butter | smooths/softens the skin |
| 14 | Lactic Acid | Solvent for the Chitosan |
| 15 | Chitosan | carrier and complexing agent for the Silver |
| | | additive |
| 16 | Limonene | oil found in citrus fruit peel. Used as a fragrance |
| 17 | Allantoin | a moisturiser with a Keratolytic effect. Used to |
| | | soften Keratin in the skin |
| 18 | Citrus Tangerina (Tangerine) | a fragrant oil of Tangerine |
| | Peel Oil | |
| 19 | Citrus Aurantium Bergamia | A fragrant oil of Bergamot |
| | (Bergamot) Fruit Oil | |
| 20 | Citrus Grandis (Grapefruit) | a fragrant oil of Grapefruit |
| | Peel Oil | |
| 21 | Citrus Aurantium Dulcis | a fragrant oil of Orange peel |
| | (Orange) Peel Oil | |
| 22 | Citral | a fragrance used again for its citrus effect |
| 23 | Linalool | another fragrance with a sweet odour used with |
| | | citrus fragrances |
| 24 | Silver Chloride | Photochemically bound to the Chitosan to render |
| | | it Antimicrobial |
| 25 | Citronellol | Mosquito repellent at short distances |
| 26 | Geraniol | Rose oil fragrance |

1. A water-based anti-microbial cosmetic preparation comprising:
0.025% to 3.2% of silver-chitosan dispersion by weight;
not more than 10% of a non-ionic surfactant by weight; and
wherein:
a ratio of non-ionic surfactant to silver chitosan by weight is at least 0.2:1;
the preparation has a pH of between 3 and 6.5; and
the preparation does not contain an ionic surfactant.

2. A cosmetic preparation according to claim 1, wherein the composition additionally comprises a pH modifier.

3. A cosmetic preparation according to claim 2, wherein the composition comprises one or more of sodium hydroxide, sodium bicarbonate, potassium hydroxide, triethanolamine, sodium phosphate, L-arginine, a high pH oil, and a high pH fragrance.

4. A cosmetic preparation according to claim 1, wherein the composition does not comprise any of a pH modifier or sodium hydroxide, sodium bicarbonate, potassium hydroxide, triethanolamine, sodium phosphate, L-arginine, a high pH oil, or a high pH fragrance.

5. A cosmetic preparation according to claim 1, wherein the non-ionic surfactant comprises one or more fatty alcohol ethoxylate, alkyl phenol ethoxylate, fatty acid alkoxylate or any non-ionic glyceryl stearate.

6. A cosmetic preparation according to claim 5, wherein the non-ionic surfactant comprises alcohol ethoxylate.

7. A cosmetic preparation according to claim 1 wherein the ratio of non-ionic surfactant to silver chitosan by weight is at least 0.5:1

8. A cosmetic preparation according to claim 7 wherein the ratio of non-ionic surfactant to silver chitosan by weight is at least 1.5:1.

9. A cosmetic preparation according to claim 1 further comprising one or more of a perfume, an oil, a viscosity modifier, a non-ionic emulsifier, glycerine, a thickener, and an emulsion stabiliser.
